(19) United States
(12) Patent Application Publication
(10) Pub. No.: US 2018/0085492 A1
(43) Pub. Date: Mar. 29, 2018

(54) ANTIOXIDANT SCAFFOLDS FOR BETA CELL DELIVERY

(71) Applicant: Northwestern University, Evanston, IL (US)

(72) Inventors: Guillermo A. Ameer, Chicago, IL (US); Xunrong Luo, Evanston, IL (US); Xiaomin Zhang, Evanston, IL (US); Yunxiao Zhu, Evanston, IL (US)

(21) Appl. No.: 15/720,403

(22) Filed: Sep. 29, 2017

Related U.S. Application Data
(60) Provisional application No. 62/401,498, filed on Sep. 29, 2016.

(51) Int. Cl. A61L 27/38; A61L 27/50; A61L 27/52; A61L 27/54; A61L 27/34

(52) U.S. Cl. CPC A61L 27/3804; A61L 27/50; A61L 27/52; A61L 2300/602; A61L 27/34; A61L 2300/64; A61L 2300/43; A61L 27/54

(57) ABSTRACT

Provided herein are thermoresponsive citrate-based hydrogels and methods of use thereof for cell delivery. In particular, pancreatic islet cells entrapped within poly(polyethyleneglycol citrate-co-N isopropylacrylamide) (PPCN) hydrogels are provided herein, as well as methods of use thereof for extra-hepatic islet transplantation.

### CROSS-REFERENCE TO RELATED APPLICATIONS

The present invention claims priority to U.S. Provisional Patent Application 62/401,498, filed Sep. 29, 2016, which is incorporated by reference in its entirety.

### FIELD

Provided herein are thermoresponsive citrate-based hydrogels and methods of use thereof for cell delivery. In particular, pancreatic islet cells entrapped within poly(polyethyleneglycol citrate-co-N isopropylacrylamide) (PPCN) hydrogels are provided herein, as well as methods of use thereof for extra-hepatic islet transplantation.

### BACKGROUND

Type 1 diabetes mellitus is a chronic condition characterized by the autoimmune-mediated destruction of pancreatic β-cells. Islets transplantation in the liver often results in limited islets survival presumably due to uneven islet distribution, abnormal blood flow dynamics and acute inflammatory response.

### SUMMARY

Provided herein are thermoresponsive citrate-based hydrogels and methods of use thereof for cell delivery. In particular, pancreatic islet cells entrapped within poly(polyethyleneglycol citrate-co-N isopropylacrylamide) (PPCN) hydrogels are provided herein, as well as methods of use thereof for extra-hepatic islet transplantation.

In some embodiments, provided herein are compositions comprising: (a) a PPCN-based hydrogel comprising citric acid, poly(ethylene glycol), glycerol 1,3-diglycerolate diacrylate, and poly-(N-isopropylacrylamide) monomers; and (b) pancreatic islet cells incorporated therein. In some embodiments, the composition comprises a carrier material of PPCN. In some embodiments, the carrier material is a composite of PPCN and one or more other materials.

In some embodiments, provided herein are compositions comprising pancreatic islet cells entrapped within a carrier material comprising poly(polyethyleneglycol citrate-co-N isopropylacrylamide) (PPCN). In some embodiments, the carrier material is a composite of PPCN and one or more additional polymer materials.

In some embodiments, provided herein are implantable systems comprising: (a) an implantable biostable retention device; (b) a carrier material comprising poly(polyethyleneglycol citrate-co-N isopropylacrylamide) (PPCN), the carrier coated onto or contained within the implantable biostable retention device; and (c) pancreatic islet cells entrapped within the carrier material. In some embodiments, the implantable biostable retention device comprises polyethylene terephthalate (PET). In some embodiments, the implantable biostable retention device comprises polyethylene glycol (PEG)-coated PET. In some embodiments, the implantable biostable retention device is a mesh, a graft, or a porous cage. In some embodiments, the implantable biostable retention device is a graft and the carrier material is coated onto a lumen of the graft. In some embodiments, the implantable biostable retention device is a porous cage and the carrier material is contained within pores of the cage. In some embodiments, the pancreatic islet cells are retained within the carrier material and/or retention device when the system is implanted in a subject. In some embodiments, soluble factors are capable of flowing into and out of the carrier material and/or retention device when the system is implanted in a subject. In some embodiments, insulin is able to flow out of the carrier material and retention device when the system is implanted in a subject.

In some embodiments, provided herein are methods of extra-hepatic islet transplantation, comprising transplanting a composition (e.g., comprising hydrogel-encapsulated islet cells) or system (e.g., comprising hydrogel-encapsulated islet cells within a retention device) into a subject. In some embodiments, provided herein are methods of treating diabetes, comprising transplanting a composition (e.g., comprising hydrogel-encapsulated islet cells) or system (e.g., comprising hydrogel-encapsulated islet cells within a retention device) into a subject.

### BRIEF DESCRIPTION OF THE DRAWINGS

FIGS. 1A-E. A. Chemical structure of cyclic RGD-functionalized-PPCN. B. liquid cRGD-PPCN (100 mg/ml) (21° C., left) and as a hydrogel above its LCST (25° C., right); C. Scanning electron microscopy image of the hydrogel's porous structure; D. Enhanced angiogenesis in the wounds that received SDF-1-PPCN (H&E staining, scale: 100 μm. E. Blood vessel density increase: PPCN-treated tissue vs. PPCN+SDF-1-treated tissue (n=20, the dashed line indicates number of blood vessels at day 0, **p<0.01, mean±SD).

FIGS. 2A-E. A. Viability of islets suspended in cell culture media or PPCN. (Scale bar: 100 μm); B. Islet transplantation onto epididymal fat pad using the cRGD-PPCN hydrogel; C. Non-fasting blood glucose levels starting at day 0 (day of transplant) through day 37 post-transplantation (cyclic RGDPPCN; Kidney control). The dashed line denotes the upper limit for normal glucose levels; D. Glucose challenge test 50 days post-transplant (Black: original PPCN); E. Immunofluorescence of the explanted fat pad.

FIGS. 3A-F. A. Digital image of the empty cage and the cage filled with PPCN. B. High magnification image of the woven structure of the cage. C. injection of the islets and PPCN into a cage that was placed on the epididymal fat pad (left). Cage containing PPCN and islets wrapped with the fat pad. D. Non-fasting blood glucose levels at days 1 and 2 post-transplantation. (cyclic RGD-PPCN; Kidney control). The dashed line denotes the upper limit for normal glucose levels; E. Glucose challenge test 50 days post-transplant; F. Cage retrieval 60 days post-transplant (Left: Fat pad w/cage wrapped inside; Right; explanted cage and the remaining tissue.

FIG. 4. Schematic of the IIDR system and digital image showing a layer of PPCN lining a lumen.

FIG. 5. Aorta interposition ePTFE graft in rat.

FIG. 6. PPCN protects the encapsulated islets from the oxidative damage, such protective effect can be prolonged to up to 12 hours (an average rang for islets harvesting and preserving before the transplantation). The controls designed in this study: Media: current standard way of culturing islets in clinics; Fibrin gel: a biocompatible nature material currently being tested for stage 1 clinical trials for islets transplantation; pNIPAAm: a negative control thermoresponsive hydrogel that does not have the same biocompatibility as PPCN.

FIG. 7. The quantification of images from FIG. 6 demonstrates that PPCN protects the islets from oxidative damage for up to 12 hours.

FIG. 8. Oxidative damage study. Freshly harvested islets were treated with lenti-viral vector encoding the RoGFP gene, a reporter gene that has a shift in excitation wavelength when oxidized. Monitoring the excitation wavelength shift provides a method to monitor the oxidation status of transduced islets. After three days of viral vector treatment, the islets were redistributed into different culture conditions (media, fibrin gel, pNIPAAm gel and PPCN gel), and treated with 100 μM H₂O₂ to induce the oxidative damage. Confocal images of the islets were taken at each time point as shown in FIG. 6. The quantification (FIG. 7) was done based on the ratio between 400/488 nm fluorescent intensity.

FIG. 9. The hydrogel islets transplant system controls blood glucose. Using 200 isletes (2 donor per recipient) the euglycemia condition was reached the day after the transplantation and is maintained until islets graft explant.

FIG. 10. Glucose tolerance tests demonstrate that transplanted islets graft responds to a sudden increase of the glucose, and control the glucose level back to the normal level within two hours.

FIG. 11. Experiments reducing the number of donor islets (e.g., 100 (1 donor per recipient) and 70 (less than 1 donor per recipient) to achieve the similar glucose control. Euglycemia status was achieved around two weeks after the transplant, and maintained until graft explant. A glucose tolerance test at 1 month after the transplant demonstrated that these transplanted islets were fully functional.

FIG. 12. Further reduction in transplant number (50 islets) showed reduced effectiveness.

FIG. 13. Histology images for the explanted islets graft from the 100 islets transplantation group (FIG. 11), fully functional islets (insulin positive) was observed between the PPCN hydrogel and the native fat pad tissue. Blood vessels were also observed around the islets providing blood flow to the region. No additional inflammation was observed at the region.

FIG. 14A-B. C₁₃ spectra of (A) virgin PET mesh with no coating, and (B) PEG hydrogel coated PET mesh.

FIG. 15. S₂ₚ spectra of a PEG hydrogel coated PET mesh and a virgin PET mesh with no coating.

FIG. 16. SEM for the modified and unmodified PET mesh.

FIG. 17. Masson's trichrome staining of the implanted PET mesh “Cage”, comparing between the before and after the PEG-modification. (black arrow indicates the fibrosis capsule formation).

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an islet cell” is a reference to one or more islet cells and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “substantially all,” “substantially complete” and similar terms refer to greater than 99%; and the terms “substantially none,” “substantially free of,” and similar terms refer to less than 1%.

The term “about” allows for a degree of variability in a value or range. As used herein, the term “about: refers to values within 10% of the recited value or range (e.g., about 50 is the equivalent of 45-55).

As used herein, the term “biocompatible” refers to materials and agents that are not toxic to cells or organisms. In some embodiments, a substance is considered to be “biocompatible” if its addition to cells in vitro results in less than or equal to approximately 10% cell death, usually less than 5%, more usually less than 1%.

As used herein, the term “biostable” refers to compositions or materials that do not readily break-down or degrade in a physiological or similar aqueous environment. For example, a material may be considered biostable if it does not degrade over the timescale of intended use.

As used herein, “biodegradable” as used to describe the polymers, hydrogels, and/or wound dressings herein refers to compositions degraded or otherwise “broken down” under exposure to physiological conditions. In some embodiments, a biodegradable substance is a broken down by cellular machinery, enzymatic degradation, chemical processes, hydrolysis, etc. In some embodiments, a wound dressing or coating comprises hydrolyzable ester linkages that provide the biodegradability.

As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

As used herein, the term “hydrogel” refers to a three-dimensional (3D) crosslinked network of hydrophilic polymers that swells, rather than being dissolved, in water.

As used herein, the term “thermoresponsive” refers to material that exhibit altered physical characteristics at different temperature ranges. Particularly relevant herein are “phase-transitioning thermoresponsive” materials. Phase-transitioning thermoresponsive” materials are soluble or in a liquid state at a first temperature range (e.g., below 26° C.) and insoluble or in a solid state at a second temperature range (e.g., 30-45° C.).

As used herein, the term “composite” refers to a material comprising two or more molecular, polymeric, and/or supramolecular constituents that are miscible with one another, and may form a single homogeneous material. While covalent connections (e.g., crosslinks) between the constituent components may be present, they are not required to form or maintain the composite or its homogeneity; rather, non-covalent and/or mechanical/physical interactions and associations are responsible for stabilizing the composite.

### DETAILED DESCRIPTION

Provided herein are thermoresponsive citrate-based hydrogels and methods of use thereof for cell delivery. In particular, pancreatic islet cells entrapped within poly(polyethyleneglycol citrate-co-N isopropylacrylamide) (PPCN) hydrogels are provided herein, as well as methods of use thereof for extra-hepatic islet transplantation.

Experiments conducted during development of embodiments herein demonstrated the capacity for stromal cell derived factor-1 (SDF-1)-PPCN to induce angiogenesis in diabetic mice, setting the foundation for using this system to maintain islet function in large animal models. In vitro, PPCN preserved the normal islet morphology while islets cultured in cell culture media began to lose this morphology due to cell spreading. The negative charge and hydrophilicity distribution within PPCN as well as its biocompatibility (low to no inflammation) created an environment that supports the viability and function of the islets, allowing successful extrahepatic islet transplantation in this mouse model. The observed restoration of euglycemia is superior to what has been reported for other hydrogels when taking into account the number of islets required to achieve euglycemia and the time it took to reach the euglycemic state.

In some embodiments, systems, methods, and compositions herein provide a platform vehicle for extrahepatic islet transplantation. PPCN allows the modification of the islet microenvironment to achieve functional vascularization and optionally display peptides (e.g., cRGD), thus enhancing long-term islet survival and function.

In some embodiments, described herein are pancreatic islet cells entrapped within the thermoresponsive citrate-based hydrogel poly(polyethyleneglycol citrate-co-N isopropylacrylamide) (PPCN), and the use of such systems in extra-hepatic islet transplantation. In some embodiments, the thermoresponsive citrate-based hydrogels (e.g., PPCN) provide a suitable microenvironment to support islets viability and function. In some embodiments, provided herein are transplantation devices comprising a stable polymeric material with pores for containing the PPCN-entrapped islet cells. In some embodiments, the compositions, devices, and methods herein provide treatment for type 1 diabetes via islet transplantation.

In some embodiments, transplantable cells (e.g., pancreatic islet cells) are maintained within a hydrogel material to facilitate transplantation of the cells into a subject. In some embodiments, the hydrogel prevents the escape of the transplantable cells into the subject, while allowing exchange of soluble factors between the subject and the cells to maintain the viability of the cells (e.g., O₂ exchange, etc.) and to benefit the subject (e.g., insulin delivery from cells to subject).

In some embodiments, transplantable cells are maintained and/or provided in a hydrogel comprising a single polymer type. In some embodiments, the hydrogel comprises multiple polymer types. In some embodiments, the polymer(s) are biodegradable. In some embodiments, the polymer(s) are biostable. In some embodiments, a combination of biostable and biodegradable polymers are utilized.

In some embodiments, the hydrogel material comprises one or more polymer-based components. In some embodiments, the hydrogel comprises a synthetic polymer selected from a polyester, poly(diol citrate) (e.g., butanediol, hexanediol, octanediol, decanediol, dodecanediol, hexadecanediol, etc.), poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(D,L-lactide), poly(caprolactone), poly(trimethylene carbonate), polyester amide, or co-polymers or composites thereof. In some embodiments, the hydrogel comprises a natural polymer, such as polysaccharides and proteins. Non-limiting examples of suitable polysaccharides include starch, amylose, amylopectin, cellulose, arabinoxylan, chitin, chitinosan, pectin, alginate, carageenan, dextrin, gums (e.g., arabic gum, gellan gum, guar gum, locust bean gum, xanthan gum), or combinations thereof. Examples of suitable proteins include but are not limited to serum albumin, egg albumin, casein, collagen, gelatin, soy protein, whey protein, zein, or combinations thereof. In some embodiments, the hydrogel comprises a combination of natural polymers, synthetic polymers, and/or other components (e.g., fillers, small molecules, peptides, crosslinkers, etc.).

In some embodiments, the hydrogel comprises a thermoresponsive polymer material. In some embodiments, the thermoresponsive polymer is the citrate-based hydrogel, poly(polyethyleneglycol citrate-co-N isopropylacrylamide) (PPCN). In some embodiments, PPCN provides a suitable microenvironment to support cell (e.g., islet) viability and function. In some embodiments, the polymer based material comprises a PPCN hydrogel. PPCN allows for the encapsulation of transplantable cells (e.g., islet cells). In some embodiments, PPCN allows for entrapment and/or supply of soluble factors to preserve the function of the embedded cells. In some embodiments, PPCN allows for release of factors (e.g., insulin) produced by the embedded cells. PPCN has intrinsic antioxidant activity. In some embodiments, PPCN allows for the successful encapsulation of islet cells and exchange of soluble factors to preserve the islet function and deliver insulin and other factors from the cells.

In some embodiments, compositions herein comprise PPCN. In some embodiments, PPCN or another polymer comprises comprise at least 0.1% citric acid monomers (e.g., >0.1%, >0.2%, >0.5%, >1%, >2%, >3%, >4%, >5%, >10%, >20%, >30%, >40%, >50%, >60%, >70%, >80%, >90%, >95%, >98%, >99%). In some embodiments, polymers herein comprise less than 99% citric acid monomers (e.g., <99%, <98%, <95%, <90%, <80%, <70%, <60%, <50%, <40%, <30%, <20%, <10%, <5%, <4%, <3%, <2%, <1%, <0.5%,). In some embodiments, polymers comprise about 99%, about 98%, about 95%, about 90%, about 80%, about 70%, about 60%, about 50%, about 40%, about 30%, about 20%, about 10%, about 5%, about 4%, about 3%, about 2%, about 1%, or about 0.5% citric acid monomers.

In some embodiments, PPCN or another polymer comprises comprise at least 0.1% polyethylene glycol monomers (e.g., >0.1%, >0.2%, >0.5%, >1%, >2%, >3%, >4%, >5%, >10%, >20%, >30%, >40%, >50%, >60%, >70%, >80%, >90%, >95%, >98%, >99%). In some embodiments, polymers herein comprise less than 99% polyethylene glycol monomers (e.g., <99%, <98%, <95%, <90%, <80%, <70%, <60%, <50%, <40%, <30%, <20%, <10%, <5%, <4%, <3%, <2%, <1%, <0.5%,). In some embodiments, polymers comprise about 99%, about 98%, about 95%, about 90%, about 80%, about 70%, about 60%, about 50%, about 40%, about 30%, about 20%, about 10%, about 5%, about 4%, about 3%, about 2%, about 1%, or about 0.5% polyethylene glycol monomers.

In some embodiments, PPCN or another polymer comprises at least 0.1% glycerol 1,3-diglycerolate diacrylate monomers (e.g., >0.1%, >0.2%, >0.5%, >1%, >2%, >3%, >4%, >5%, >10%, >20%, >30%, >40%, >50%, >60%, >70%, >80%, >90%, >95%, >98%, >99%). In some embodiments, polymers herein comprise less than 99% glycerol 1,3-diglycerolate diacrylate monomers (e.g., <99%, <98%, <95%, <90%, <80%, <70%, <60%, <50%, <40%, <30%, <20%, <10%, <5%, <4%, <3%, <2%, <1%, <0.5%). In some embodiments, polymers comprise about 99%, about 98%, about 95%, about 90%, about 80%, about 70%, about 60%, about 50%, about 40%, about 30%, about 20%, about 10%, about 5%, about 4%, about 3%, about 2%, about 1%, or about 0.5% glycerol 1,3-diglycerolate diacrylate monomers.

In some embodiments, PPCN or another polymer comprises at least 0.1% N-isopropylacrylamide monomers (e.g., >0.1%, >0.2%, >0.5%, >1%, >2%, >3%, >4%, >5%, >10%, >20%, >30%, >40%, >50%, >60%, >70%, >80%, >90%, >95%, >98%, >99%). In some embodiments, polymers herein comprise less than 99% N-isopropylacrylamide monomers (e.g., <99%, <98%, <95%, <90%, <80%, <70%, <60%, <50%, <40%, <30%, <20%, <10%, <5%, <4%, <3%, <2%, <1%, <0.5%). In some embodiments, polymers comprise about 99%, about 98%, about 95%, about 90%, about 80%, about 70%, about 60%, about 50%, about 40%, about 30%, about 20%, about 10%, about 5%, about 4%, about 3%, about 2%, about 1%, or about 0.5% N-isopropylacrylamide monomers.

In some embodiments, the PPCN-based materials described herein are liquid at sub-physiologic temperatures (e.g., 36° C., 35° C., 34° C., 33° C., 32° C., 31° C., 30° C., 29° C., 28° C., 27 ° C., 26° C., 25° C., 24° C., 23° C., 22° C., 21° C., 20° C., or lower or ranges therebetween). In some embodiments, the PPCN-based materials described herein gel at or near physiologic temperatures (e.g., 30° C., 31° C., 32° C., 33° C., 34° C., 35° C., 36° C., 37° C., 38° C., 39° C., 40° C., or ranges therebetween).

In some embodiments, compositions herein comprise a composite of PPCN and one or more additional components (e.g., polymer components). In some embodiments, a composite material comprises at least 1% (e.g., >>1%, >2%, >3%, >4%, >5%, >10%, >20%, >30%, >40%, >50%, >60%, >70%, >80%, >90%, >95%, >98%, >99%) PPCN or PPCN-based polymer. In some embodiments, a composite material comprises less than 99% (e.g., <99%, <98%, <95%, <90%, <80%, <70%, <60%, <50%, <40%, <30%, <20%, <10%, <5%, <4%, <3%, <2%, <1%) PPCN or PPCN-based polymer. In some embodiments, composites comprise one or more polymereic materials, in addition to PPCN or a PPCN-based polymer. Suitable polymers for use in such composites include, but are not limited to: collagen, elastin, hyaluronic acid and derivatives, sodium alginate and derivatives, chitosan and derivatives gelatin, starch, cellulose polymers (for example methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextran and derivatives, polysaccharides, poly(caprolactone), fibrinogen, poly(hydroxyl acids), poly(L-lactide) poly(D,L lactide), poly(D,L-lactide-co-glycolide), poly(L-lactide-co-glycolide), copolymers of lactic acid and glycolic acid, copolymers of ε-caprolactone and lactide, copolymers of glycolide and ε-caprolactone, copolymers of lactide and 1,4-dioxane-2-one, polymers and copolymers that include one or more of the residue units of the monomers D-lactide, L-lactide, D,L-lactide, glycolide, ε-caprolactone, trimethylene carbonate, 1,4-dioxane-2-one or 1,5-dioxepan2-one, poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids), and copolymers of the above polymers as well as blends and combinations of the above polymers. (See generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986; herein incorporated by reference in their entireties).

In some embodiments, polymers and composites are obtained and/or prepared according to standard techniques and or those described herein or incorporated by reference. In some embodiments, the hydrogel-entrapped islet cell compositions described herein are reversibly implanted in a subject. In some embodiments, reversible implantation allows the implanted material/device to be retrieved after a period of time (e.g., 1 day, 2 days, 3 days, 4 days, 5 days, 6 days, 1 week, 2 weeks, 3 weeks, 1 month, 2 months, 3 months 4 month, 5 months, 6 months, 1 year, 2 years, 3 years, 4 years, or more, or ranges therebetween). In some embodiments, the retrievability of the hydrogel-islet systems herein is achieved by including it in a porous “sealed cage.” In some embodiments, the sealed cage comprises a biostable (e.g., nondegradable) polymer (e.g., polyethylene terephthalate, nylon, silk, etc.). The islets-hydrogel mixture is entrapped within the cage. (e.g., within the pores of the cage material). After transplantation, the cage sits as a physical barrier between the transplanted islets and the surrounding native tissue while its porous structure still allows functional fluid/nutrient exchange and neo-vascular formation.

In some embodiments, the islet-hydrogel system is placed as a coating within the lumen of a vascular graft, said coating used as a basis to seed endothelial cells, for example, to create an endothelium on the hydrogel-islets. In some embodiments, the resulting intravascular islets graft is a non-obstructive vascular interposition graft that enhances the supply of oxygen to the islets. In some embodiments, this system provides surgeons with options regarding the anastomotic locations within the body's vascular system that facilitates one or more of the following: create the least disturbance to the native blood flow, allow easy implant and explant, maximize islet functionality (e.g., due to improved oxygenation, due to protection from deleterious blood flow dynamics, etc.), etc.

In some embodiments, cages, grafts, meshes, or other devices for containing the hydrogel-islet compositions are non-biodegradable. Some non-limiting examples of suitable nondegradable materials include polymeric materials, for example, polyolefins such as polyethylene (including ultra high molecular weight polyethylene) and polypropylene including atactic, isotactic, syndiotactic, and blends thereof; polyethylene glycols; polyethylene glycol (PEG) coated polyethylene terephthalate (PET), polyethylene oxides; polyisobutylene and ethylene-alpha olefin copolymers; fluorinated polyolefins such as fluoroethylenes, fluoropropylenes, fluoroPEGSs, and polytetrafluoroethylene; polyamides such as nylon, Nylon 6, Nylon 6,6, Nylon 6,10, Nylon 11, Nylon 12, and polycaprolactam; polyimines; polyesters such as polyethylene terephthalate, polyethylene naphthalate, polytrimethylene terephthalate, and polybutylene terephthalate; polyethers; polybutester; polytetramethylene ether glycol; 1,4-butanediol; polyurethanes; acrylic polymers; methacrylics; vinyl halide polymers such as polyvinyl chloride; polyvinyl alcohols; polyvinyl ethers such as polyvinyl methyl ether; polyvinylidene halides such as polyvinylidene fluoride and polyvinylidene chloride; polychlorofluoroethylene; polyacrylonitrile; polyaryletherketones; polyvinyl ketones; polyvinyl aromatics such as polystyrene; polyvinyl esters such as polyvinyl acetate; etheylene-methyl methacrylate copolymers; acrylonitrile-styrene copolymers; ABS resins; ethylene-vinyl acetate copolymers; alkyd resins; polycarbonates; polyoxymethylenes; polyphosphazine; epoxy resins; aramids; rayon; rayon-triacetate; spandex; silicones; and copolymers and combinations thereof.

In some embodiments, cages, grafts, or other devices comprise poses that contain he hydrogel-islet material. In some embodiments, the pore size prevents the outflow of the islets from the hydrogel. In some embodiments, the hydrogel material prevents the outflow of the islet cells. In some embodiments, the pore size prevents the outflow of undegraded hydrogel. In some embodiments, the pore size prevents outflow of isltes after degradation of the hydrogel. In some embodiments, the cages, grafts, or other devices allow the system, or undegraded portions thereof, to be retrieved from a subject after or during use.

The islets of Langerhans are the regions of the pancreas that contain the endocrine (e.g., hormone-producing) cells (e.g., beta cells). In some embodiments, provided herein are implantable systems comprise clusters of islet cells within a hydrogel material. In some embodiments, islet cells are transferred from culture (e.g., in a Transwell plate) to an aggregation or cluster forming vessel (e.g., conical well (e.g., AggreWell (e.g., U.S. Pub. No. 2011/0086375; U.S. Pub. No. 2010/0068793; U.S. Pub. No. 2012/0149051; herein incorporated by reference in their entireties)), etc.). In some embodiments, clustered islet cells are entrapped with the hydrogel materials or composites described herein for transplantation into a subject.

Provided herein are methods and systems for treating a patient suffering from a conditions or disease (e.g., diabetes (e.g., Type 1 diabetes, Type 2 diabetes), etc.). In certain embodiments, methods involve implanting the islet cells within a hydrogel carrier (e.g., further within a biostable device) cells into the patient. If appropriate, cells are co-administered with one or more pharmaceutical agents or bioactives that facilitate the survival and function of the transplanted cells. These agents may include, for example, insulin, members of the TGF-β family, including TGF-β 1, 2, and 3, bone morphogenic proteins (BMP-2, -3, -4, -5, -6, -7, -11, -12, and -13), fibroblast growth factors-1 and -2, platelet-derived growth factor-AA, and -BB, platelet rich plasma, insulin growth factor (IGF-I, II) growth differentiation factor (GDF-5, -6, -7, -8, -10, -15), vascular endothelial cell-derived growth factor (VEGF), pleiotrophin, endothelin, among others. Other pharmaceutical compounds can include, for example, nicotinamide, glucagon like peptide-I (GLP-I) and II, GLP-1 and GLP-2 mimetibody, Exendin-4, retinoic acid, parathyroid hormone, MAPK inhibitors, etc.

### Experimental

### EXAMPLE 1

### PPCN synthesis

Poly(polyethyleneglycol citrate-co-N-isopropylacrylamide) (PPCN) was synthesized from citric acid, poly(ethylene glycol), glycerol 1,3-diglycerolate diacrylate (and/or itaconic acid) and poly-N-isopropylacrylamide.

For example, Citric acid, polyethylene (PEG), and glycerol 1,3-diglycerolate diacrylate in a 5:9:1 molar ratio are reacted in a polycondensation reaction at 140° C. for 45 minutes by melting under constant stirring to produce poly(polyethyleneglycol citrate) acrylate prepolymer (PPCac). For free radical polymerization, PPCac and N-isopropylacrylamide (NIPAAm) are added to a three-necked flask in a 1:1 weight to weight ratio and dissolved in 1,4-dioxane. AIBN radical initiator is added to the PPCac and NIPAAm mixture (final concentration: 6.5×10⁻³ M) and reacted for 8 hours at 65° C. in a nitrogen atmosphere.

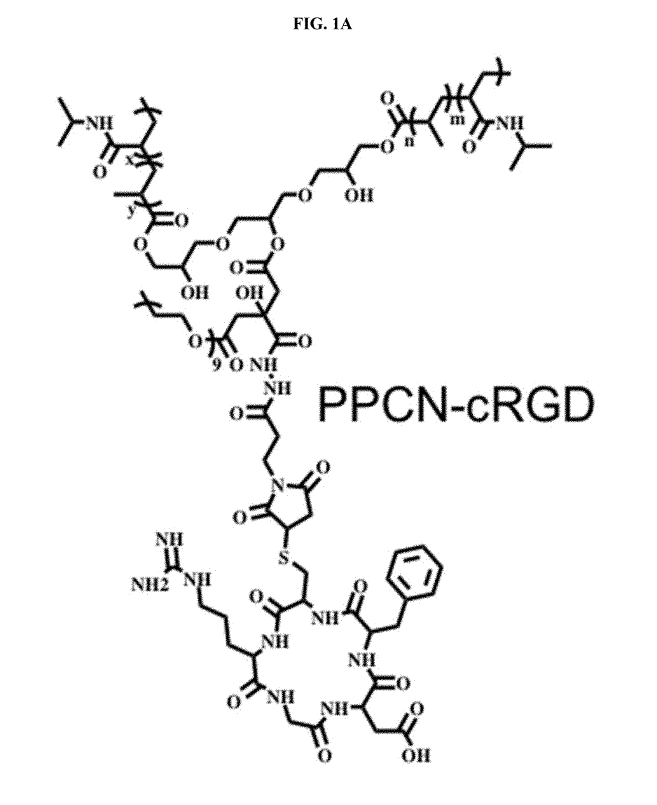

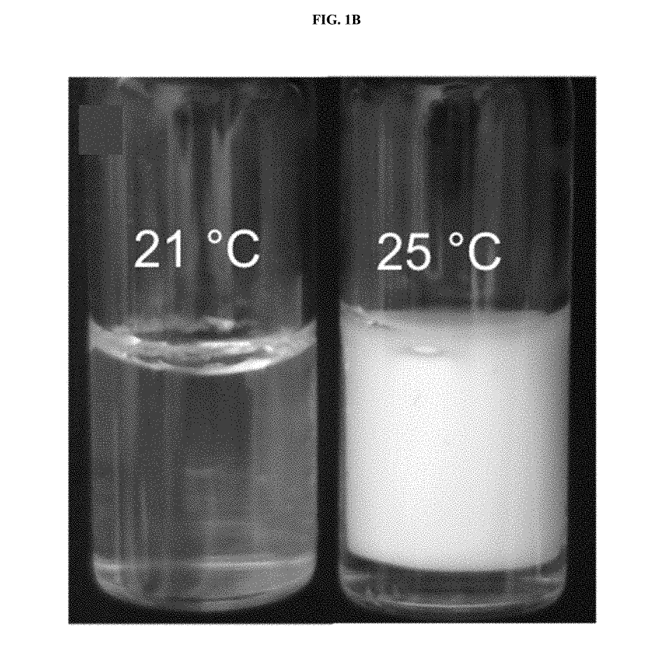

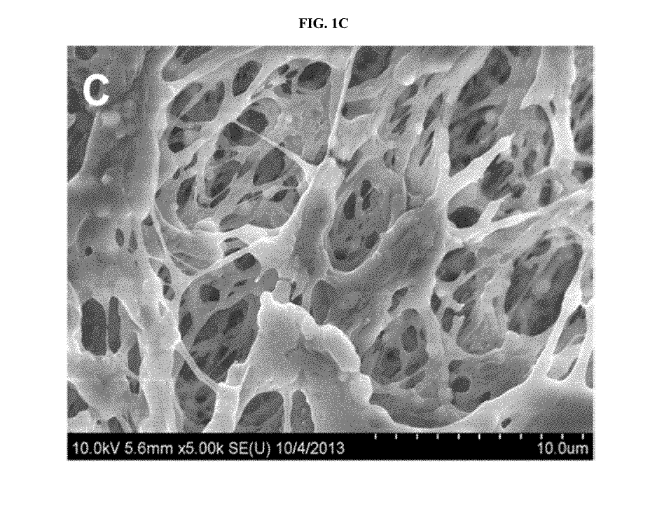

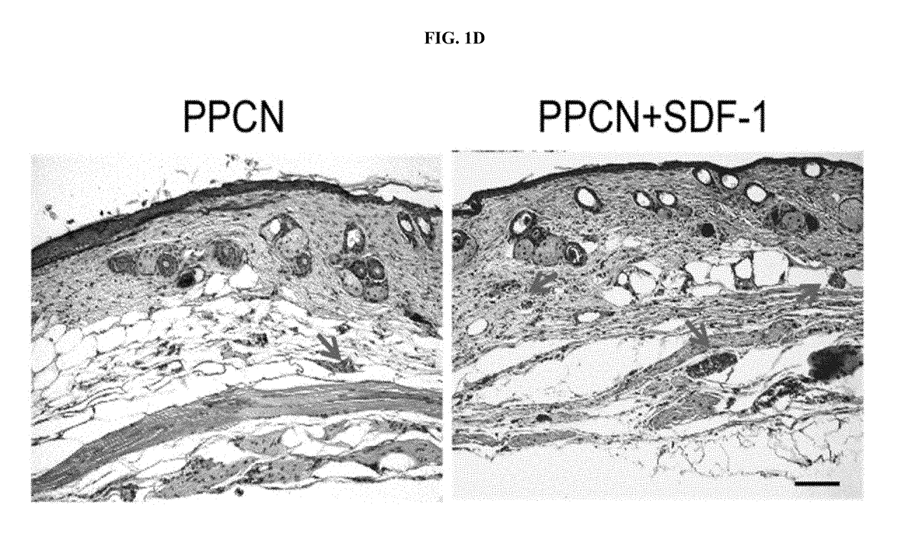

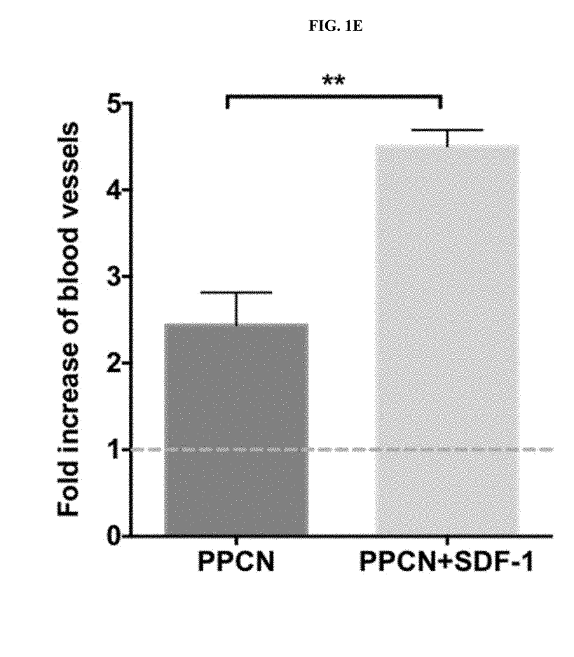

The PPCN copolymer was dissolved in 1,4-dioxane and purified by precipitation in diethyl ether and vacuum dried. PPCN is gas sterilized using ethylene oxide. The resulting polymer was further functionalized with cell adhesion peptides through a hetero-bifunctional linker N-β-maleimidopropionic acid hydrazide (FIG. 1A) or used to entrap stromal cell derived factor-1 (SDF-1). The robust angiogenic capacity of SDF-1-releasing PPCN was evaluated in a diabetic mouse wound healing model (FIG. 1D, 1E).

### EXAMPLE 2

### cRGD-Linked to PPCN

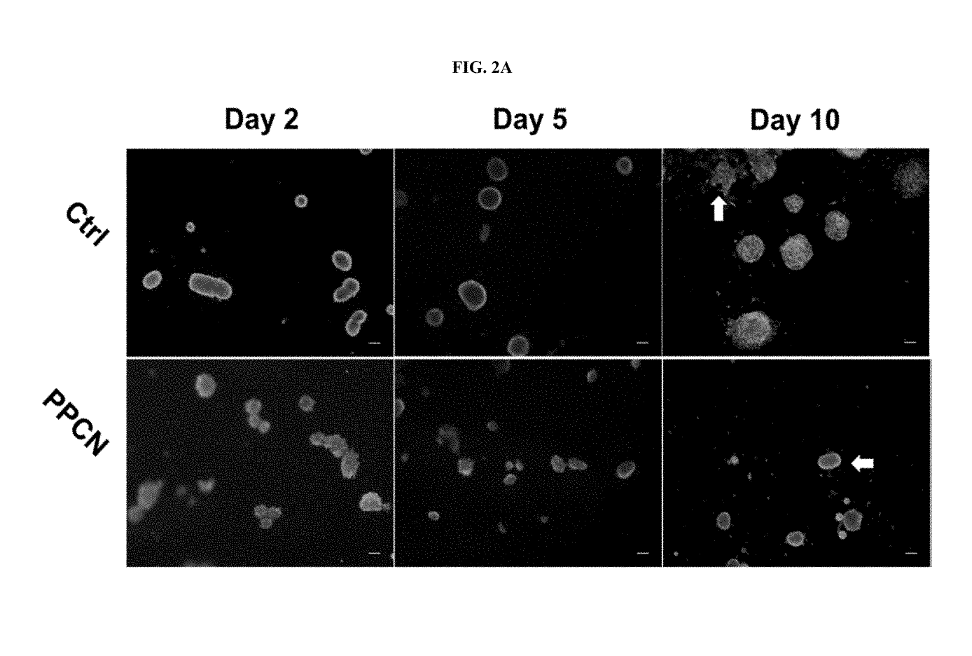

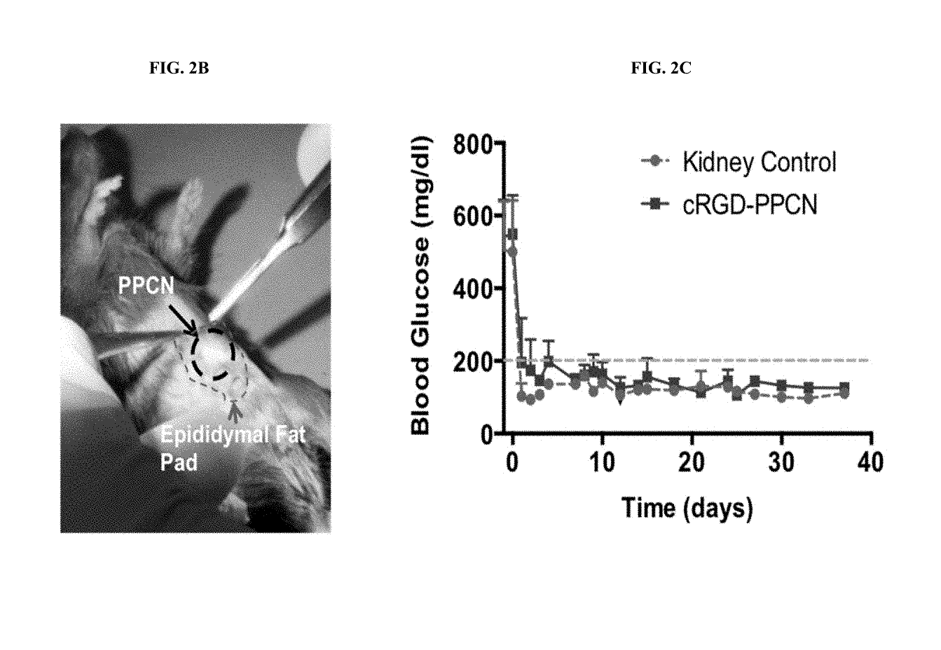

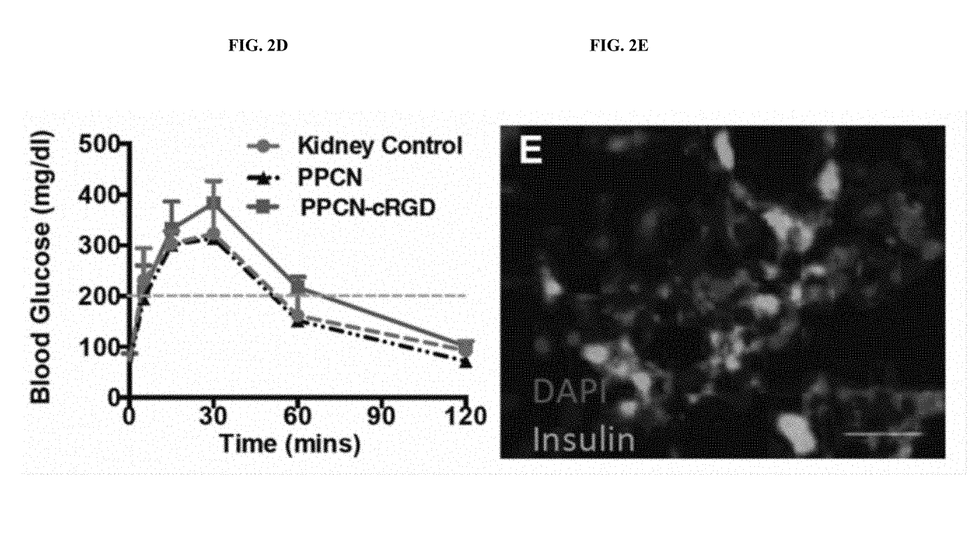

Cyclic-RGD (cRGD) was covalently linked to PPCN and the cRGD-PPCN retained its ability to reversibly change between liquid and gel phases at a lower critical solution temperature (LCST) of 25° C. and maintain micro- and macroporosity within the gel phase. SDF-1 was entrapped and slowly released from PPCN. Wounds treated with SDF-1-PPCN demonstrated enhanced vascularization (FIG. 1). Ten days after isolation, Islets entrapped within PPCN maintained their original morphology and viability in vitro. Islets entrapped in cRGD-PPCN restored euglycemia (glucose<200 mg/dl) within an average of 1.8 (±0.8) days following transplantation. Euglycemia was maintained in the cRGD-PPCN and control group for the duration of the experiment (37 days) (FIG. 2). Good islet viability was observed in vitro with a Live/Dead Assay (FIG. 2A) and the in vivo islets function was assessed using the streptozotocin-induced diabetic mice model (FIG. 2B). 200 mouse islets were isolated from a syngeneic donor and either: a) applied with PPCN hydrogel only onto the epididymal fat pad or b) directly transplanted into the kidney capsule (control). Non-fasting glucose levels were monitored over time to assess the performance of the islets post-transplantation. The islets grafts were explanted 69 days post-transplantation and the hyperglycemia post explant verified the key function of the graft (FIG. 2C). The intraperitoneal glucose tolerance test (IPGTT) was performed at 50 days post-transplantation, the matching trend between the kidney control and the original PPCN and PPCN-cRGD groups represents the active function of the transplanted islets in respond to the blood glucose change (FIG. 2D). Immunofluorescence was performed on the explanted grafts, positive staining of the insulin further confirms the presence of the functional islets within the graft (FIG. 2E).

### EXAMPLE 3

### Exemplary Islets/PPCN-Loaded PET Cage

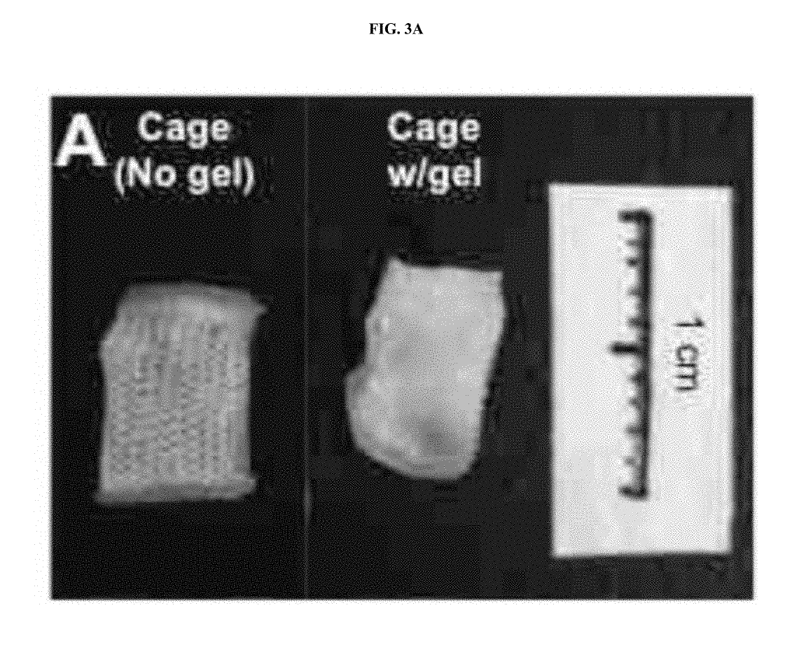

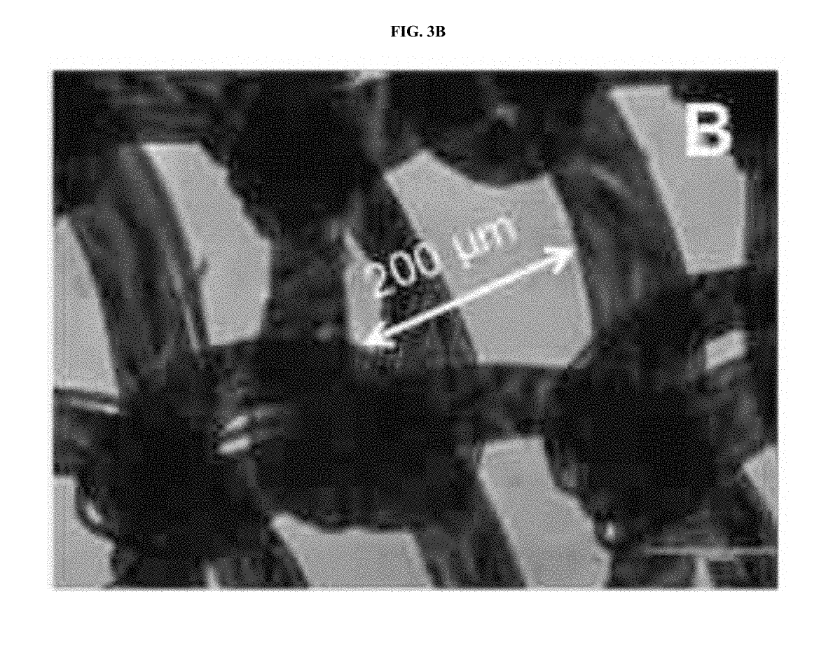

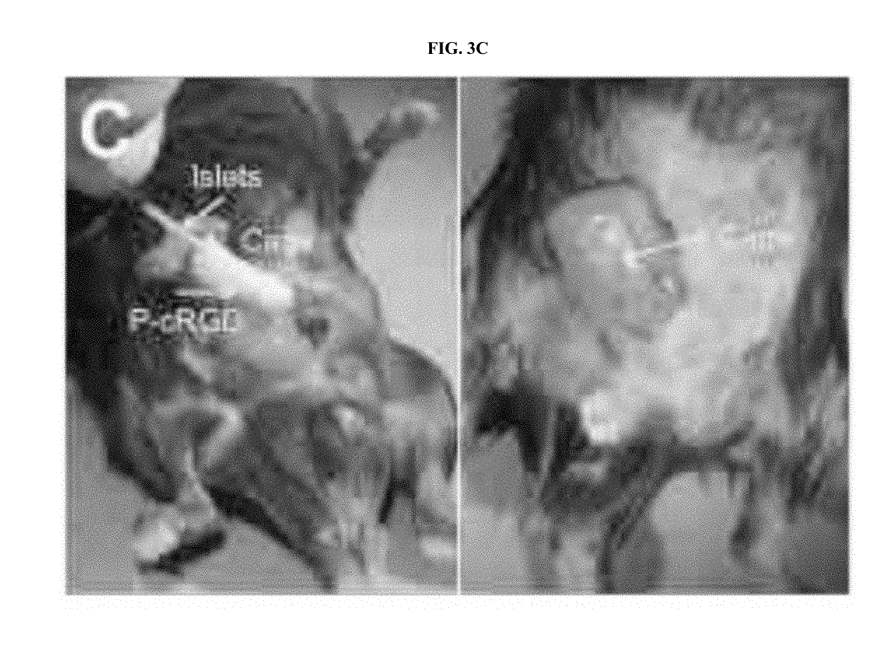

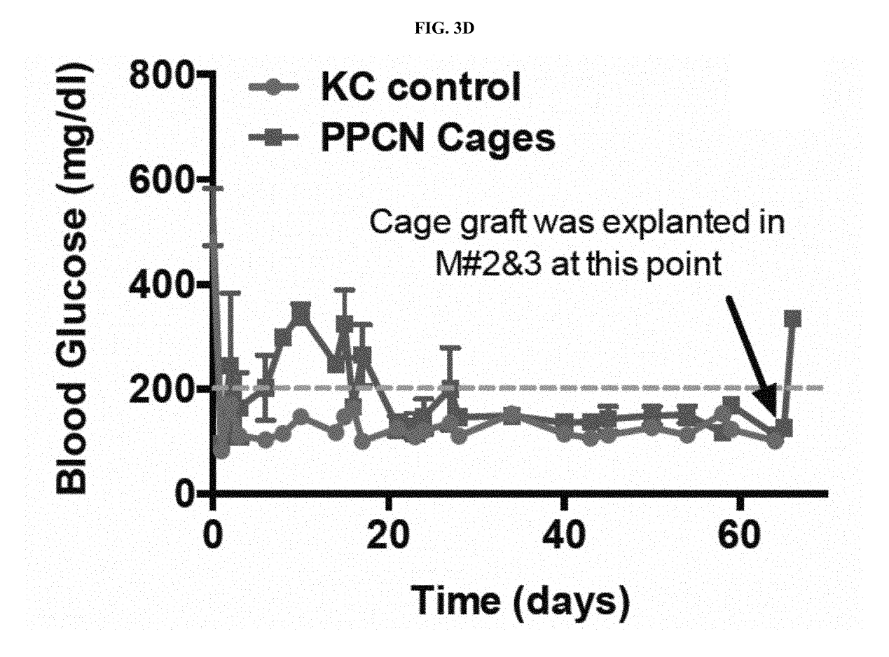

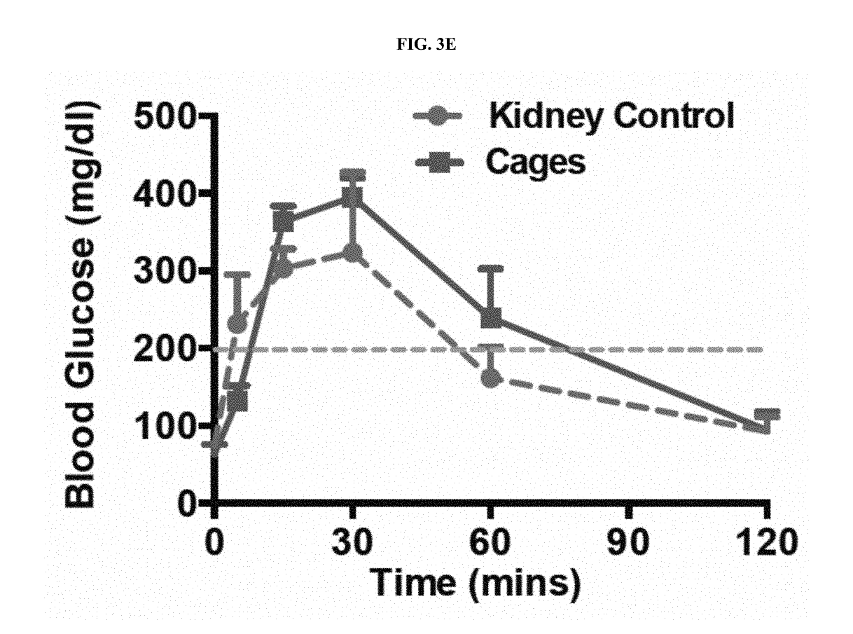

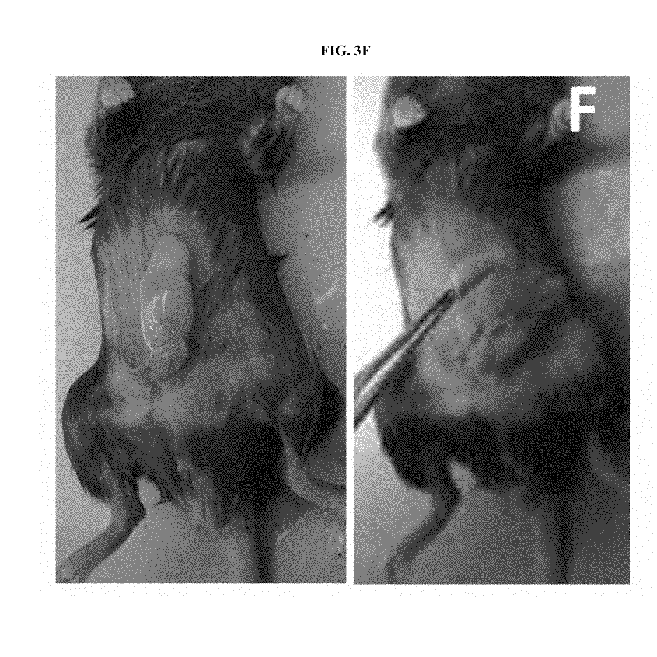

An exemplary cage is made out of polyethylene terephthalate (PET) with an average pore size of 150 μm (FIG.3A, 3B). During the transplantation, the islets were suspended in PPCN before injected into the cage on epididymal fat pad (FIG. 3C). Non-fasting blood glucose levels were monitored, normal stable glycaemia level was achieved around 20 days post-transplantation, the graft function in stabilizing the glucose level was verified by the recovery of the hyperglycemia states after explanting the graft 64 days post-transplantation (FIG. 3D). While the overall rising and lowering trend are very similar, the IPGTT test presents a slightly delayed glucose response in cage group when comparing to the kidney controls (FIG. 3E). This delay indicates that further modification of the cage shape and material are needed to improve the function of the encapsulated islets. The retrevability of the cages were verified during the graft explant at day 64 post-transplantation as shown in FIG. 3F.

### EXAMPLE 4

### Exemplary Intravascular Islet Transplantation System

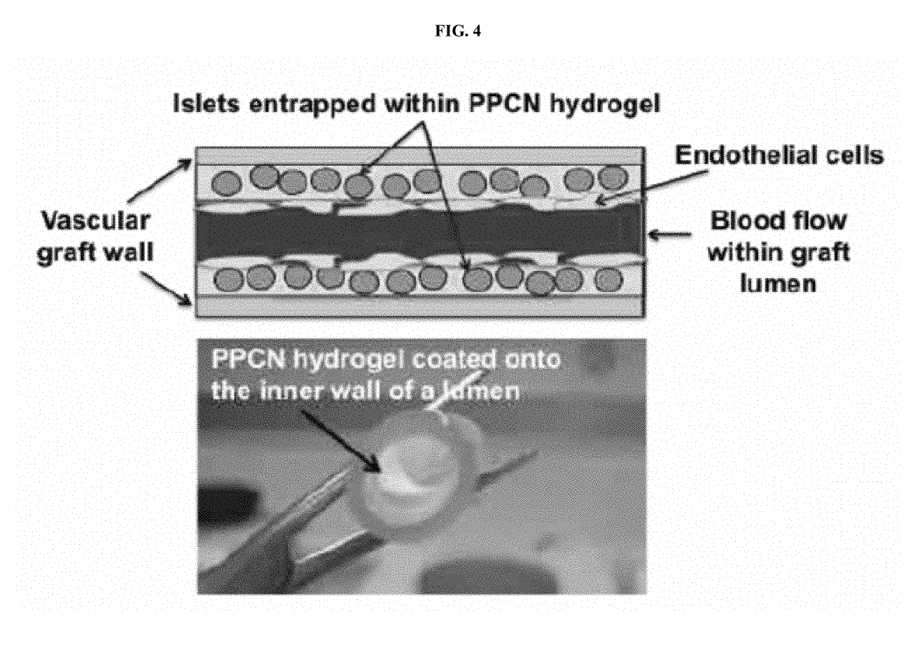

PPCN is located concentrically within the lumen of the vascular graft in a non-obstructive fashion. The vascular graft is either synthetic or biologic (e.g., tissue engineered or decellularized tissue). The design of the IIDR system allows the islets to be in close proximity to flowing blood without direct contact as the islets will be embedded throughout PPCN coated onto the graft wall in the lumen and separated by an endothelial cell monolayer (FIG. 4). A technique that gels PPCN onto the vascular graft walls is used, and has been used with an aorta graft interposition model in rat (FIGS. 4 & 5).

### EXAMPLE 5

### Oxidative Environment Effects on Islets and the Ability of the Polymer to Protect the Islets

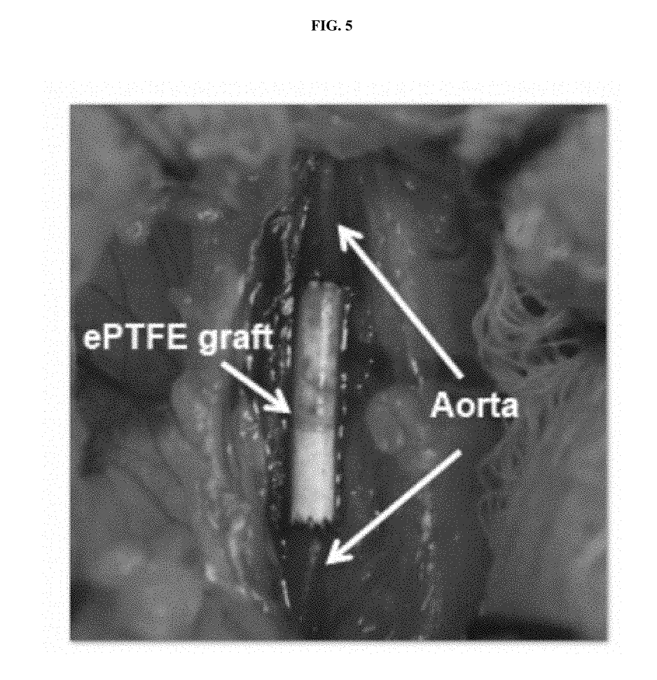

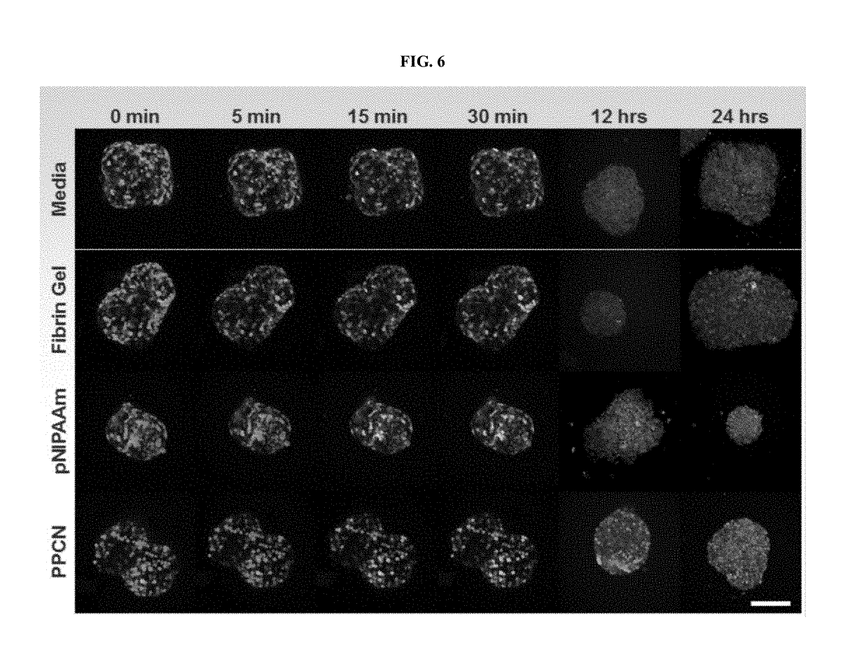

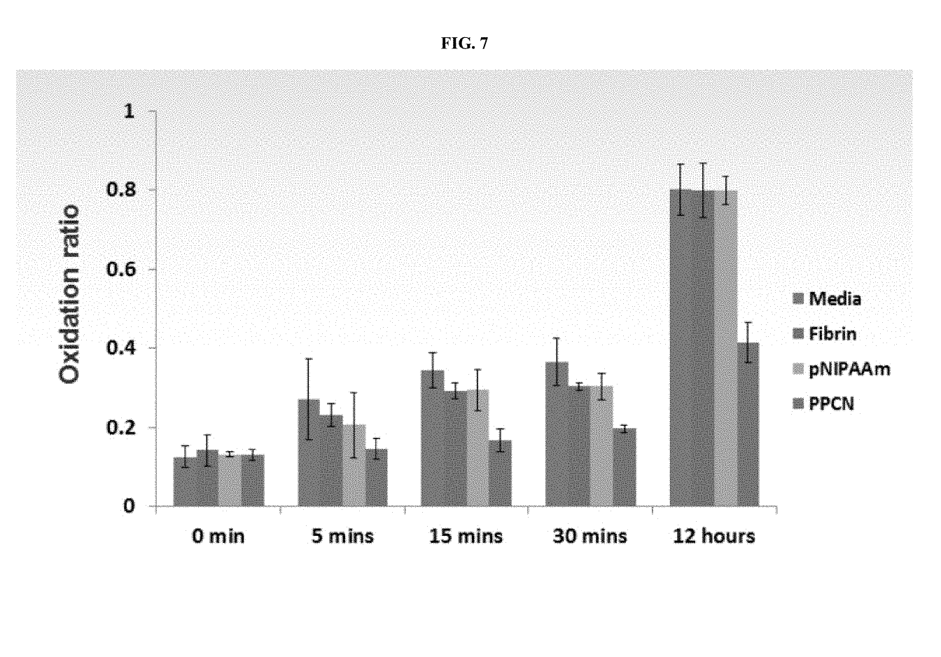

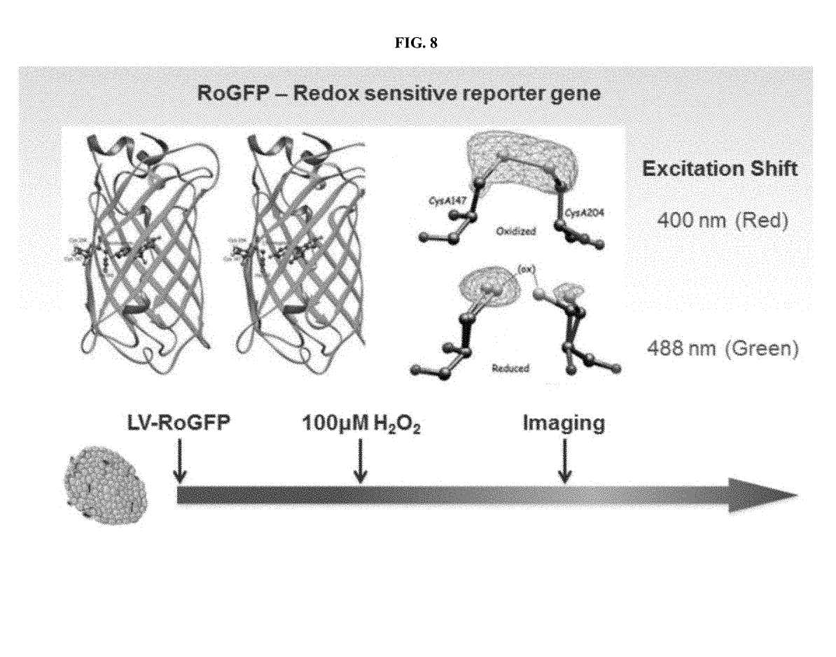

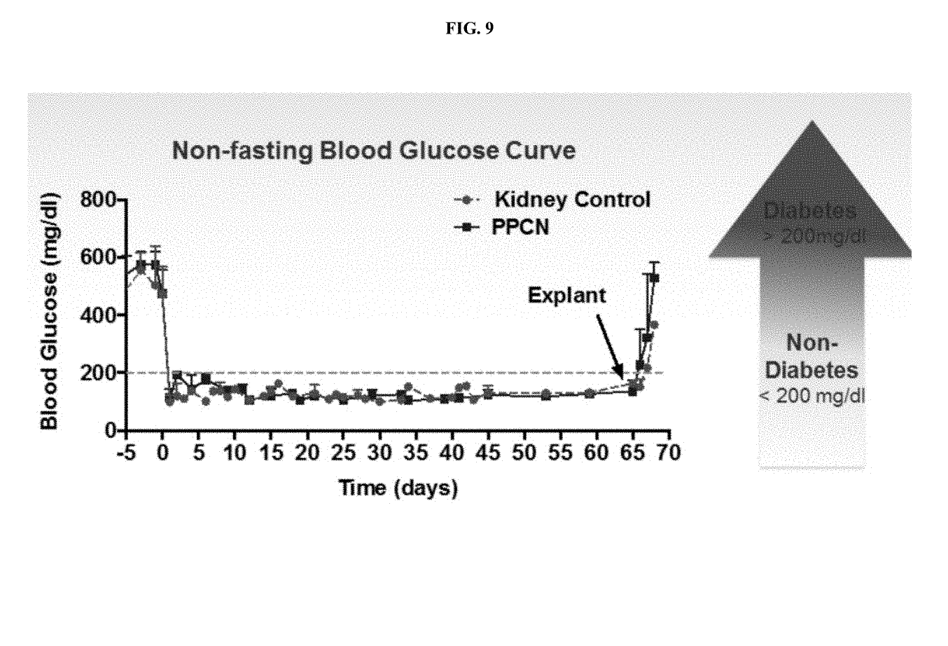

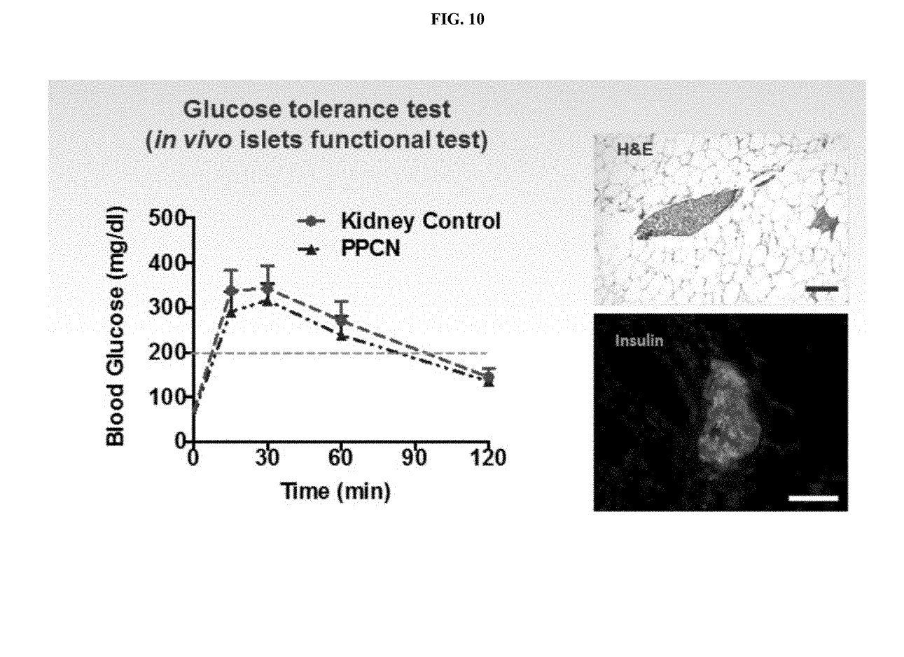

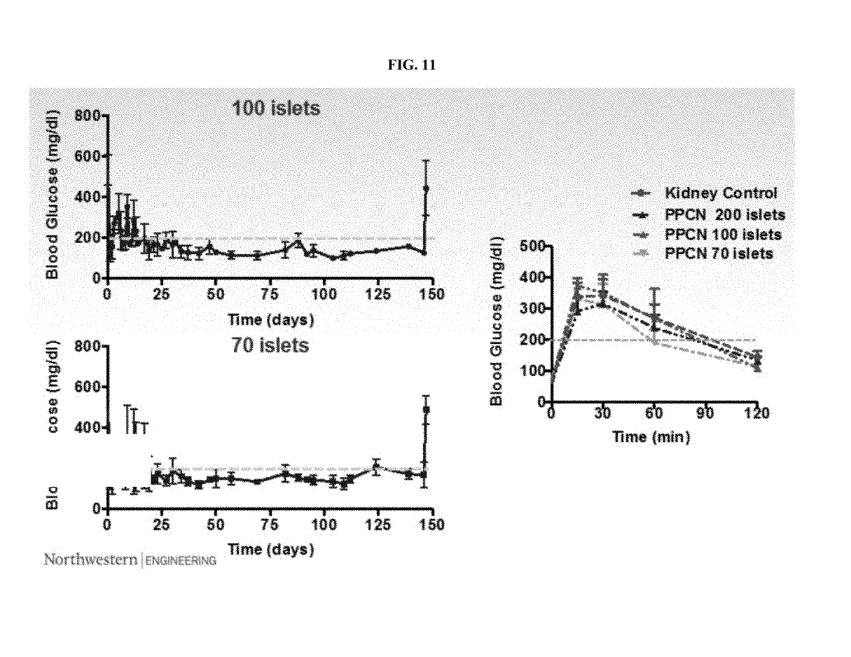

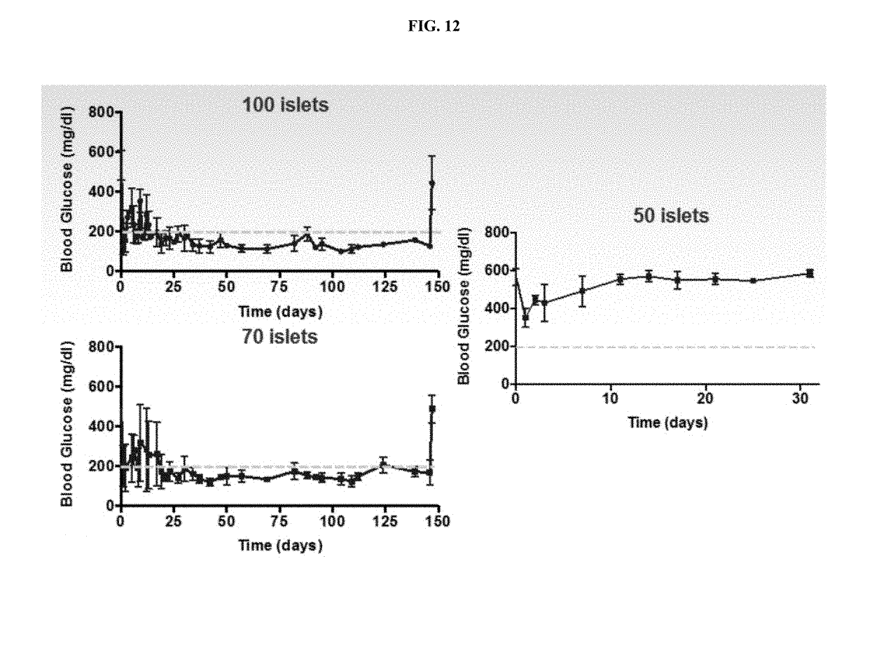

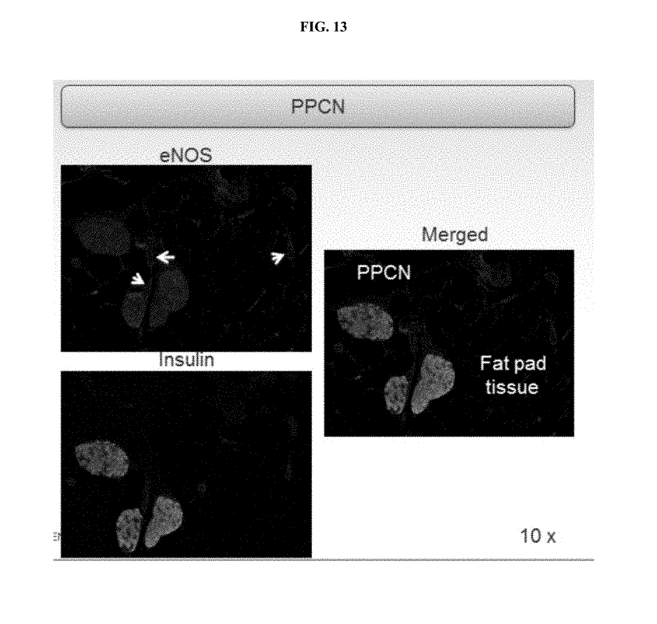

Freshly harvested islets were treated with lenti-viral vector encoding the RoGFP gene. RoGFP is a reporter gene that has a shift in excitation wavelength when oxidized. Monitoring the excitation wavelength shift provides a non-destructive method to monitor the oxidation status of transduced islets. After three days of viral vector treatment, the islets were redistributed into different culture conditions (media, fibrin gel, pNIPAAm gel and PPCN gel), and treated with 100 μM H₂O₂ to induce the oxidative damage. Confocal images of the islets were taken at each time point as shown in FIG. 6. The quantification (FIG. 7) was done based on the ratio between 400/488 nm fluorescent intensity.

### EXAMPLE 6

### PEG Modified PET Mesh Resulted in Significant In Vivo Fibrosis Reduction

Methods to make polyethylene glycol (PEG) coated polyethylene terephthalate (PET) mesh were modified from Scott et al (Biomaterials 29.34 (2008): 4481-493; incorporated by reference in its entirety). PET mesh (0.18 mm thick, 0.05 mm pore size, gift from SurgicalMesh™ Brookfield, Conn.) was cut into 1 cm×2 cm pieces. The meshes were then functionalized using air-plasma etching via radio frequency glow discharge (RFGD). They were treated for 20 min (10 min each side) using 100% power. The Harrick PDC-32G plasma etcher was used for RFGD.

PEG hydrogels were formed using Michael-type addition by first combining 8-arm PEG Amine (MW 10,000, tripentaerythritol core) with 8-arm PEG Vinylsulfone (MW 10,000, tripentaerythritol core) at 200 mg/mL in phosphate buffered saline (PBS). Both 8-arm PEGs were purchased from Jenkem Technology. The solution was mixed at 37° C. for 6 h, rotating at 40 rpm. After the 6 hours, the RFGD-PEG mesh was inserted into the solution, and was rotated for another 12 hours at 37° C. After the 12 hours, the meshes were removed from the hydrogel and stored in PBS at room temp.

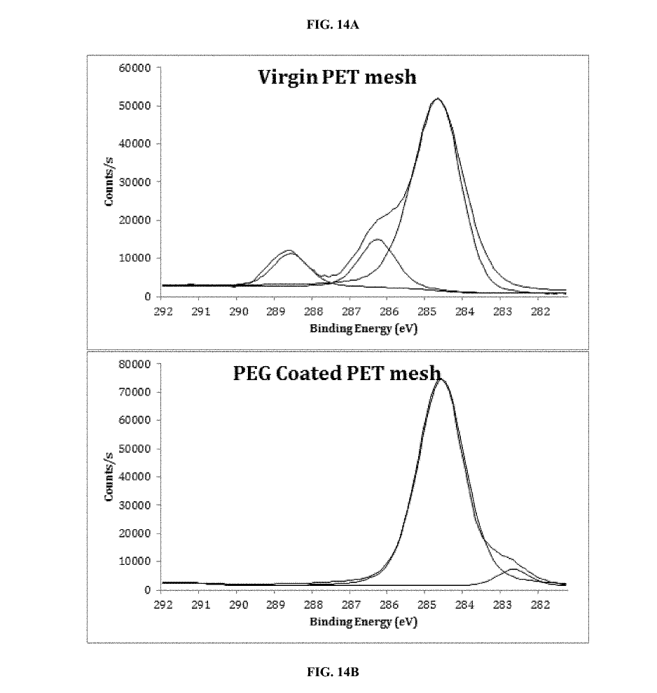

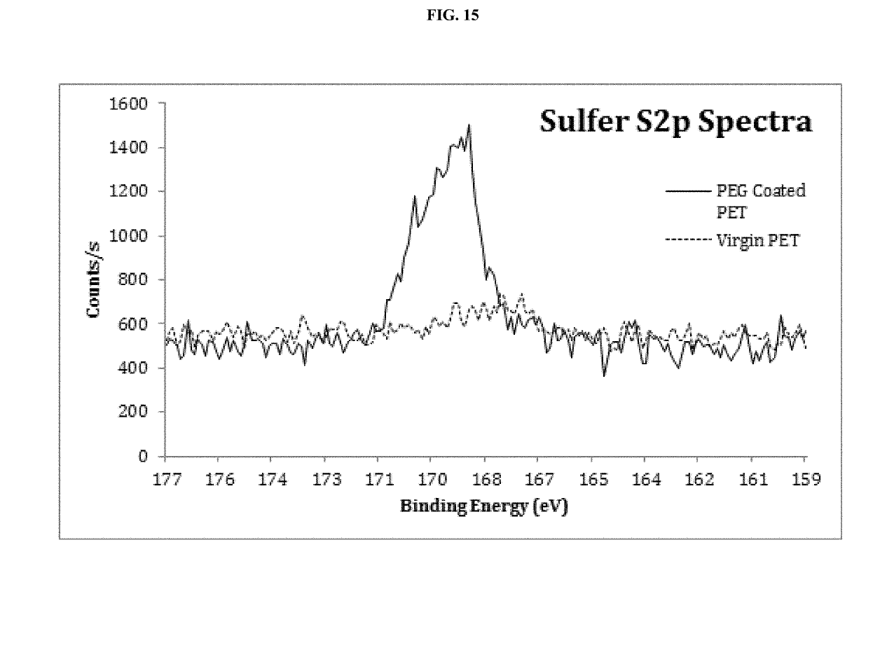

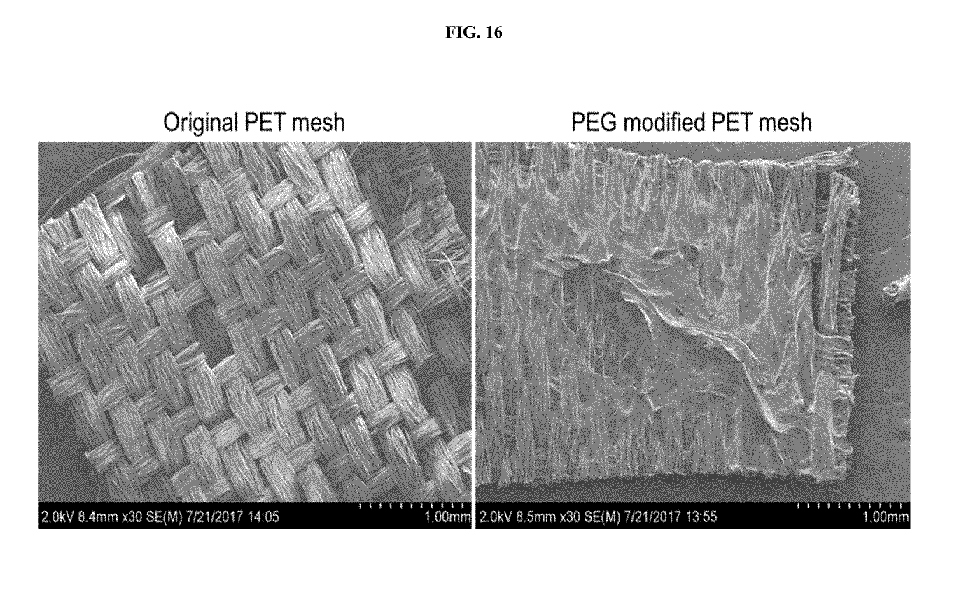

The mesh was characterized by x-ray photoelectron spectroscopy (XPS) using the Thermo Scientific ESCALAB 250Xi. C₁ₛ spectra show a clear drop in —C—O-(˜286.40) and —C═O (˜288.10) bonds in the PEG hydrogel coated PET mesh, while also observing an increase in —C—C-(˜284.60) bonds (FIG. 14). An increase in the sulfur was also observed in S₂ₚ spectra accounting for the vinyl sulfone-amine bonds (FIG. 15). The coating was also verified by SEM, the coating layer could be observed when comparing to the unmodified controls. (FIG. 16)

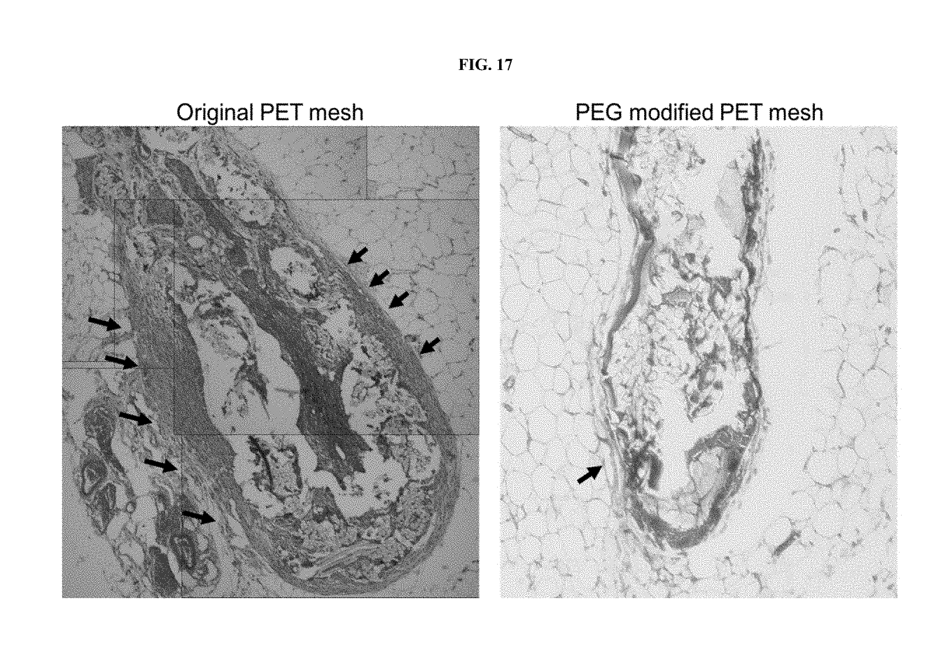

Before implantation, the surface characteristic of the meshes were cut into ˜4 mm circles and sterilized in ethanol. Sterilized meshes were implanted into the fat pad of 3, C57BL/B6 mice. Control PET meshes were implanted on the spleen side of the mouse while PEG coated meshes were implanted on the liver side of the mouse. The meshes were explanted 19 days later, sectioned and stained for Masson's trichrome. Significant decrease of fibrosis capsule was observed when comparing the new modified meshes to the non-modified ones. (FIG. 17).

1. A composition comprising pancreatic islet cells entrapped within a carrier material comprising poly(polyethyleneglycol citrate-co-N isopropylacrylamide) (PPCN).

2. The composition of claim 1, wherein the carrier material is a composite of PPCN and one or more additional polymer materials.

3. An implantable system comprising:
(a) an implantable biostable retention device;
(b) a carrier material comprising poly(polyethyleneglycol citrate-co-N isopropylacrylamide) (PPCN), the carrier coated onto or contained within the implantable biostable retention device; and
(c) pancreatic islet cells entrapped within the carrier material.

4. The implantable system of claim 3, wherein the implantable biostable retention device comprises polyethylene terephthalate (PET).

5. The implantable system of claim 4, wherein the implantable biostable retention device comprises polyethylene glycol (PEG)-coated PET

6. The implantable system of claim 3, wherein the implantable biostable retention device is a mesh, a graft, or a porous cage.

7. The implantable system of claim 6, wherein the implantable biostable retention device is a graft and the carrier material is coated onto a lumen of the graft.

8. The implantable system of claim 6, wherein the implantable biostable retention device is a porous cage and the carrier material is contained within pores of the cage.

9. The implantable system of claim 3, wherein the pancreatic islet cells are retained within the carrier material and/or retention device when the system is implanted in a subject.

10. The implantable system of claim 3, wherein soluble factors are capable of flowing into and out of the carrier material and/or retention device when the system is implanted in a subject.

11. The implantable system of claim 10, wherein insulin is able to flow out of the carrier material and retention device when the system is implanted in a subject.

12. A method of extra-hepatic islet transplantation, comprising transplanting a system of claim 3 into a subject.

13. A method of treating diabetes, comprising transplanting a system of claim 3 into a subject.
